(19) United States
(12) Patent Application Publication
(10) Pub. No.: US 2010/0029885 A1
(43) Pub. Date: Feb. 4, 2010

(54) CROSSLINKED POLYMER SHEETS AND METHODS FOR MAKING SUCH

(75) Inventor: Petrus Henricus Maria Van Kessel, Tilburg (NL)

Correspondence Address: BROWDY AND NEIMARK, P.L.L.C.;624 NINTH STREET, NW, SUITE 300, WASHINGTON, DC, 20001-5303, US

(73) Assignee: FUJIFILM Manufacturing Europe B.V., Tilburg (NL)

(21) Appl. No.: 12/298,747

(22) PCT Filed: Apr. 27, 2007
(86) PCT No.: PCT/NL07/50186

§ 371 Date: Sep. 2, 2009

(30) Foreign Application Priority Data
Apr. 27, 2006 (EP) 06113202.3

(51) Int. Cl. C07K 1/107; B29C 41/00

(52) U.S. Cl. 527/200; 264/299

(57) ABSTRACT

The invention relates to sheet materials comprising crosslinked polymer based layers and production methods thereof. More particularly the invention relates to gelatin based layers that are hardened by an amine-amine crosslinking compound such as triazine type crosslinking compounds. Gelatins solutions used for such materials are buffered to control the hardening speed of the gelatin solutions. This invention also relates to applications of such hardened gelatin layers such as image recording materials but also applications such as test-substrates applicable for pharmaceutical or cosmetic industry.

### FIELD OF THE INVENTION

The present invention relates to sheet materials comprising layers that are made by crosslinking dissolved polymers and production methods thereof. In particular, this invention relates to gelatin layers crosslinked by hardeners such as triazine hardeners. Further this invention relates to applications of sheet materials such as image recording materials but also applications such as test-substrates applicable for pharmaceutical or cosmetic industry.

### BACKGROUND

Gelatin is used in a variety of applications. In photographic emulsions gelatin is used as a dispersing medium for silver halide crystals and for preparing oil-in-water emulsions. More recently, gelatin is also applied in inkjet recording materials as image receiving medium or as a medium for accommodating high solvent loads from the inkjet inks. Gelatin also has pharmaceutical, cosmetic and medical applications. Artificial skin for example is prepared from gelatin and also test substrates for testing pharmaceutical and cosmetic compounds.

Such uses of gelatin have in common that the structural integrity of gelatin layers is achieved by crosslinking gelatin molecules. A lot of studies have been done on crosslinking or hardening of gelatin by the photographic industry, where gelatin is a vital component of the photographic emulsion layers. In the Theory of the Photographic process (T. H. James et al, 4^{th} edition 1977, chapter 2, section III) the hardening of gelatin is described, and various types of hardeners are mentioned. A more recent study on hardening of gelatin with various hardeners is described for example by Sakvarelidze et al (Russian Journl of Applied Chemistry, 2003, 76(7), 1143-1151).

The use of hardened gelatin layers in inkjet applications is mentioned in for example EP 594896 wherein gelatin is mixed with other polymers, and is preferably crosslinked. S-triazines are mentioned as possible crosslinking compounds. Other publications applying crosslinked gelatin for inkjet recording media are for example EP 0716929; EP 0856414; EP 029,108; U.S. Pat. No. 3,889,270; EP 0701902; EP 1569802; WO 2004/110774.

CS 141238 discloses gelatin sheets for use as artificial skin that have increased tensile strength after crosslinking with trimethoxytriazine. Use of crosslinked gelatin in artificial skin is also disclosed in for example JP 59013210, U.S. Pat. No. 4,522,753, PL 133551, EP 0331786, EP 0411124, U.S. Pat. No. 4,971,954, EP 0440198, JP 04129563, EP 0568334, EP 0702081, WO 00/09018, WO 04/28547.

For gelatin, there is a vast number of known crosslinking agents—also known as hardening agents. Examples of crosslinking compounds include aldehyde compounds such as formaldehyde and glutaraldehyde, ketone compounds such as diacetyl- and chloropentanedion, bis(2-chloroethylurea), 2-hydroxy-4,6-dichloro-1,3,5-triazine, reactive halogen-containing compounds disclosed in U.S. Pat. No. 3,288,775, carbamoyl pyridinium compounds in which the pyridine ring carries a sulphate or an alkyl sulphate group disclosed in U.S. Pat. No. 4,063,952 and U.S. Pat. No. 5,529,892, divinylsulfones, and the like.

S-Triazine derivatives are well known crosslinking compounds. 2-Hydroxy-4,6-dichloro-s-triazine is a derivative commonly used in photographic application. It has the drawback that it is unstable. WO 00/15619 discloses the production of a stabilized 2-hydroxy-4,6-dichloro-s-triazine to which (hydrogen)carbonate buffer is added during production. Other buffers are added to suppress formation of carbon dioxide during storage.

It is a well known phenomenon in the art that by hardening gelatin microgels are formed which can cause defects on a coated substrate, e.g. a web, such as spots, lines or streaks. Such microgels are typically formed at the addition point of crosslinking compound into a coating solution, in dead spaces in the supply lines of the coater or in dead spaces of the coater itself. After prolonged production times such microgels will have grown sufficiently to be released and deposited onto the coater or the web causing defects.

EP 1367436 acknowledges that the hardening speed of a coated gelatin is critical for the above described properties and describes a coating fluid comprising at least 1 wt % gelatin and 1-200 effective micromoles crosslinking compound per gram of coating fluid with reduced hardening speed, so that more reactive crosslinking compounds or higher molecular weight gelatin can be used. The fact that hardening already occurs immediately after mixing of crosslinking compound with a gelatin containing coating fluid is mentioned. It can be expected that the problem of microgel formation in the coating equipment is somewhat delayed, and the document is silent with respect to problems due to slower hardening rates.

JP 05265130 attempts to solve the problem of microgel formation by coating an aqueous solution of crosslinking compound containing less than 3% gelatin on top of the conventional photographic layers. However, decreasing the crosslinking compound concentration to reduce reaction rate gives an undesired high water load that consequently reduces drying efficiency. Further, problems with crystallisation of crosslinking compound can be expected at the water/air interface, especially with lower gelatin concentration. After coating, crosslinking compound concentration in all layers should be balanced. This can only occur by diffusion of crosslinking compound from its addition point. Measures are taken so that the top layer has enough mechanical strength after hardening. For example, this is done by adding acid (pig skin) gelatin in addition to or instead of lime bone gelatin. Acid gelatin is known to have higher hardening speed than lime bone gelatin. When crosslinking compound is added on top of such a layer unbalanced hardening of the coated layers can be expected.

JP 11242305 is another attempt to solve the problem of microgel formation by coating an aqueous solution of crosslinking compound but now as the bottom layer. Also in this case crystallization can occur at the water/air interface during coating. Corrosion of the coating equipment can occur due to salts present in the crosslinking compound solution.

In spite of attempts to solve the problem of microgel formation in coating equipment for photographic recording materials there remains a need for methods of continuously coating such recipes for extended periods of time without the occurrence of defects caused by formation of microgels.

### SUMMARY OF THE INVENTION

It is an object of this invention to provide a sheet material comprising at least one crosslinked polymer layer that is essentially free from defects caused by microgels or microparticles which are formed during production of such sheet materials.

It is also an object of the invention to provide a method of producing such sheets materials by continuously coating for extended periods of time without the occurrence of defects caused by formation of microgels or microparticles.

It is also an object of the invention to provide a method for producing image recording materials with an increased run time, meaning that longer continuous coating runs can be done without stopping due to formation of microgels.

It is a further object of the invention to provide a method for continuous production of gelatin based sheets for medical or pharmaceutical applications without the risk of coating disturbances.

It is also an objective of the invention to provide gelatin sheet materials for tissue engineering such as artificial skins without defects or irregularities.

Surprisingly all objectives were met by a method for producing a sheet material comprising at least one crosslinked polymer layer on a substrate said method comprising the steps of

(i) providing a coating solution comprising between 3 and 30 weight percent of a dissolved polymer comprising at least one group selected from a hydroxy-group a thiol-group and an amine-group, and a buffering compound that does not react with the crosslinking compound and has a pKa value at 25° C. of between at least 6.5 and at most 8.5
(ii) adjusting the pH of said coating solution to a pH at 40° C. of between 6.0 and 9.0
(iii) adding a crosslinking compound to said coating solution and
(iv) applying said coating solution onto said substrate within at most 30 minutes after addition of said crosslinking compound thus obtaining a coated layer and
(v) optionally separating said substrate from said coated layer.

A good example of a method according to the invention is crosslinking of gelatin. Hardening or crosslinking of gelatin proceeds in two steps, binding a first, and then a second lysine. The first step is fastest. The second step proceeds with much lower speed and is hence rate-determining. It is this second reaction step that has an effect on viscosity and is responsible for forming microgels.

For the group of substituted heterocyclic aromatic crosslinking compounds, comprising s-triazines, the reaction is auto-catalytic due to the strong pH dependency of the second lysine coupling. For other amine-amine crosslinking compounds the reaction rate of the second step is reduced when pH decreases.

Countermeasures that delay microgel formation when using substituted heterocyclic aromatic crosslinking compounds are aimed primarily at this second, rate determining, step. Binding of crosslinking compound to lysine in the first step releases protons, accelerating the binding of a second lysine. A person skilled in the art who wants to reduce hardening speed will simply increase the pH of the coating liquid in which crosslinking compound is added. This should delay the second reaction step. This measure however appears insufficient and even appears to counteract the process as the first reaction step, and thus the release of protons, occurs even faster at higher pH.

It is common in the art to apply crosslinking compound solutions as they are produced, without further adjustments. For example 2-hydroxy-4,6,-dichloro-s-triazines are synthesized in the presence of carbonate, thus resulting in a carbonate-buffered crosslinking compound solution. Additional buffers may be added by the manufacturers, such as borate buffers, to postpone formation of CO₂. These buffers have high pKa, generally above 9.0 to prevent the first reaction step in the solution, in which replacement of a chloride by a hydroxy group occurs. Such crosslinking compound molecules have lost the ability to crosslink gelatin molecules. Until now it has not been realized that the objectives of the crosslinking compound manufacturer, namely preventing the first reaction step, are in contradiction to the objective of controlling the second reaction step. Due to their high pKa, the buffers used by the crosslinking compound manufacturer are unsuitable for controlling the second reaction step during crosslinking.

In case of amine-amine crosslinking compounds other than substituted heterocyclic aromates, the release of protons caused by the first reaction step decreases the speed of the second reaction step. For such crosslinking compounds it is also important to prevent that pH drops too much. Thus the present inventors were pleasantly surprised that the inventive countermeasure found for the specific group of substituted heterocyclic aromates also is beneficial for amine-amine crosslinking compounds for which the pH effect on the second reaction step is exactly opposite.

A further advantage, besides preventing formation of microgels, of the present method is that the viscosity of the gelatin solution remains low for a prolonged time, resulting in more flexibility in the process of coating or otherwise processing the gelatin solution.

Another advantage is that for s-triazine crosslinking compounds that contain carbonate buffer, the formation of carbon dioxide is delayed. Formation of carbon dioxide bubbles in gelatin-solutions may cause defects in the final product.

### DESCRIPTION OF THE INVENTION

Crosslinking of polymers such as gelatins by compounds such as triazines is a pH dependent reaction. A known problem related to pH-dependent crosslinking is the formation of microparticles or microgels in dead spaces of equipment. Such particles or gels accumulate in dead space, and cause defects in a product when they are released.

According to the present invention this can be prevented by a method in which a crosslinking compound that has a pKa at 25° C. of between 6.5 and 8.5 is added to a polymer solution having a pH at 40° C. of between 6.0 and 9.0, prior to applying that solution to a substrate. Preferably the condition is met that the absolute value of (pH_{40° C.}−pKa_{25° C.})≦1.0, for which the scientific notation is |(pH_{40° C.}−pKa_{25° C.})|≦1.0. pH_{40° C.} is the pH of the solution in which the crosslinking compound is added, measured before addition of the crosslinking compound. pKa_{25° C.} is the pKa of the added crosslinking compound. Thus in one embodiment the method according tot the invention is wherein for the coating solution obtained in step (iii) described above the condition is met that |(pH_{40° C.}−pKa_{25° C.})|≦1.0.

In principle the pKa of the buffering compound at 40° C. should be taken as a starting point. However, for most buffering compounds only the pKa at 25° C. is known. Measurement or calculation of the pKa values at 40° C. is laborious. We found that a valid selection of buffering compounds is possible when comparing the pKa at 25° C. to the pH of the polymer solution at 40° C.

Several different types of crosslinking compounds are known in the art. Inorganic crosslinking compounds like chromium or aluminium salts have a pH-dependent binding to gelatin or collagen via its carboxylic groups. Organic crosslinking compounds react with the amine-groups, for example with lysine and hydroxylysines in gelatin or collagen. Such crosslinking compounds are called amine-amine crosslinking compounds. In the context of this invention, wherever lysine is mentioned this includes hydroxylysine, unless stated otherwise. Examples of organic crosslinking compound are aldehydes, ketones, carboxylic- and carbamic acid derivatives, active olefins, s-triazines, epoxides, aziridines, isocyanates, carbodiimines and isoxazolium salts, pyridinium ethers, carbamoyl- and carbamoyloxy pyridinium ions and sulfon based crosslinking compounds such as sulfonate esters and sulfonyl halides.

In one embodiment the crosslinking compound is a triazine type crosslinking compound, preferably a substituted s-triazine. The reaction between triazine crosslinking compounds and gelatin is known to be pH-dependent.

A commercially available s-triazine crosslinking compound is for example 2-hydroxy-4,6-dichloro-triazine. A method for the production of this specific triazine crosslinking compound is described in U.S. Pat. No. 6,570,011. The method involves hydrolysis of cyanuric chloride with strong lye, in the presence of a bicarbonate buffer. When the synthesis is complete, additional buffer is added, other than bicarbonate. The purpose of the buffer is the stabilization of the triazine itself during storage and transport. The 2-hydroxy-4,6-dichloro-triazine is easily hydrolyzed whereby one chloride is substituted yielding 2,4-dihydroxy-6-chloro-triazine, which has lost its crosslinking capability. Buffers other than bicarbonate are added as stabilizers in order to prevent formation of CO₂, which may be hazardous due to e.g. pressure build up in storage containers.

Buffer is added to stabilize the triazine crosslinking compound solution itself; that is, to make sure that hydrolysis of triazine is sufficiently reduced during storage and transport. Depending on the order/supply systems of both the manufacturer and the user of the crosslinking compound, time between production of the crosslinking compound and addition into a coating solution can be anytime between a few weeks to a couple of months. During this period a certain amount of crosslinking compound will have been hydrolyzed, and the released protons will have used up an unknown amount of buffer. To some extent the pH of the crosslinking compound solution can be used as a measure to determine the progress of hydrolysis. It is however not practically feasible to determine the remaining buffering capacity.

Further, in normal operation the crosslinking compound solution can be heated up to a temperature between 35 and 45 degrees Celsius before addition to the coating solution, which also normally has a temperature of between about 35 and 45 degrees Celsius. Reason is that, when locally lowering the temperature by addition of crosslinking compound, gelatin will form a gel. At higher temperature the hydrolysis speed of crosslinking compound will increase, causing further variations in remaining buffering capacity.

The stabilizing buffers added by the manufacturer are selected for a high storage capacity and have generally a high pKa value in order to prevent the first reaction step, since the crosslinking compound loses its crosslinking capability if the first leaving group of the triazine is lost.

Carbonate/bicarbonate buffers for example have a pKa of 10.3 for the first protonation step and 6.4 for the second protonation step.

Suitable buffers are those having a pKa between 6.5 and 8.5. Buffers can be inorganic or organic. The buffer must not react with the crosslinking compound to prevent that the crosslinking reaction, or hardening, is impaired or the hardening speed is affected significantly. Preferably the buffer does not react chemically with the crosslinking compound at all. A skilled person can easily determine from theory or from simple laboratory tests if a crosslinking compound and buffer will react.

In case of s-triazines for example, the buffering compound should not contain hydroxyl groups, thiol groups or amine groups, although carboxyl groups are allowed.

In case protons are released during the hardening or crosslinking reaction, the pKa of the buffer is preferably lower than the pH of the gelatin solution containing the hardener. In case protons are released preferably (pH_{40° C.}−pKa_{25° C.})≦1.0, more preferably −0.5≦(pH_{40° C.}−pKa_{25° C.})≦1.0 and most preferably 0.25≦(pH_{40° C.}−pKa_{25° C.})≦0.75. In case protons are consumed during the hardening reaction the pKa of the buffer is preferably higher than the pH of the gelatin solution containing the hardener. Whe protons are consumed preferably (pKa_{25° C.}−pH_{40° C.})≦1.0, more preferably −0.5≦(pKa_{25° C.}−pH_{40° C.})≦1.0 and most preferably 0.25≦(pKa_{25° C.}−pH_{40° C.})≦0.75.

The above preferences can be combined by referring to the absolute values of the difference between pH and pKa so that preferably |(pH_{40° C.}−pKa_{25° C.})|≦1.0, more preferably −0.5≦|(pH_{40° C.}−pKa_{25° C.})|≦1.0 and most preferably 0.25≦|(pH_{40° C.}−pKa_{25° C.})|≦0.75

Some examples of suitable inorganic buffers are given in the table below:

| Acid | pKa (298K) |
|---|---|
| H₂PO₄⁻ | 7.2 |
| HSO₃⁻ | 7.2 |
| HClO | 7.5 |
| Pb(H2O)ₙ²⁺ | 7.8 |
| Cu(H2O)₄²⁺ | 8.0 |
| HBrO | 8.7 |

Suitable organic buffers having a pKa between 6.5 and 8.5 is given in the table below, which is not meant to be a complete overview:

| Acid | Acronym | pKa (298K) |
|---|---|---|
| N-(2-acetamido)iminodiacetic acid | ADA | 6.6 |
| 3-(N-Morpholino)-2-hydroxypropanesulfonic acid | MOPSO | 6.8 |
| N-(2-acetamido)-2-aminethanesulfonic acid | ACES | 6.9 |
| Imidazole | | 7.0 |
| Piperazine-N,N′-bis(2-ethanesulfonic acid) | PIPES | 7.1 |
| N,N-Bis(2-hydroxyethyl)-2-aminoethanesulfonic acid | BES | 7.3 |
| 3-(N-Morpholino)propanesulfonic acid | MOPS | 7.3 |
| 3-[N-(Trishydroxymethyl)methylamino]-2- | TAPSO | 7.5 |
| hydroxypropanesulfonic acid | | |
| N-Tris(hydroxymethyl)methyl-2-aminoethanesulfonic acid | TES | 7.6 |
| 3-(N-Morpholino)butanesulfonic acid | MOBS | |
| N-2-hydroxyethylpiperazine-N′-2-ethanesulfonic acid | HEPES | 7.7 |
| N-Ethyl-morpholine | | 7.7 |
| N-hydroxyethylpiperazine-N′-2-hydroxypropanesulfonic acid | HEPPSO | 7.8 |
| Tri ethanol amine | | 7.8 |
| Piperazine-N,N′-bis2-hydroxypropane sulfonic acid | POPSO | 7.8 |
| N-Tris(hydroxymethyl)methylglycine | TRICINE | 7.9 |
| Trimethylol aminomethane | TRIS | 8 |
| Glycylglycine | | 8.1 |
| N,N-Bis(2-hydroxyethyl)glycine | BICINE | 8.2 |
| N-Tris(hydroxymethyl),ethyl-3-aminopropanesulfonic acid | TAPS | 8.2 |

Suitable organic buffers to be applied in the present method in combination with substituted aromatic crosslinking compounds, preferably with s-triazines, are those that have a pKa at 25° C. of between 6.5 and 8.5 and have a structure I:

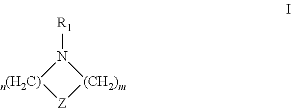

wherein
Z=carbon, oxygen, sulphur or N—R₂
2≦(n+m)≦6
R₁, R₂=—(—CH₂—)_{q}-A
A=hydrogen, —SO₃H, —COOH, —PO₃H₂ or a salt thereof
A is other than hydrogen for at least one of R1 and R2 in structure I, and
q=1-4 and if A=—COOH then q=0-4.

Also compounds with general structure II are suitable provided that their pKa at 25° C. is between 6.5 and 8.5.

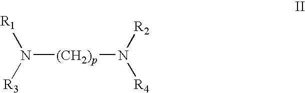

wherein
R₁, R₂, R₃ and R₄=—(—CH₂—)_{q}-A
A=hydrogen, —SO₃H, —COOH, —PO₃H₂ or a salt thereof
A is other than hydrogen for at least one of R₁, R₂, R₃ and R₄
for at least two of R₁, R₂, R₃ and R₄ A is other than hydrogen
q=1-4 and in case A=—COOH then q=0-4
1≦p≦3

In one embodiment of the present method the buffering compound is selected from the group consisting of structure I and structure II as depicted above. In one embodiment the buffering compound has structure I as depicted above. In another embodiment the buffering compound is a phosphate. Suitable phosphate buffer compounds are potassium- or sodium phosphates. In one embodiment of the present method the amount of buffering compound is at least 0.01 mol per mol of crosslinking compound.

Preferred non-limiting examples of suitable buffers are:

| Acid | Acronym | pKa (298K) |
|---|---|---|
| 3-(N-Morpholino)propanesulfonic acid | MOPS | 7.3 |
| Piperazine-N,N′-bis(2-ethanesulfonic acid) | PIPES | 7.1 |

The purpose of adding the buffer according to the invention is to allow the first reaction step between crosslinking compound and gelatin to proceed and to delay the second reaction step by preventing a fast pH drop due to the release of protons in the first reaction step. The reaction needs to be delayed only in the liquid phase. After coating a layer, or forming a sheet, the reaction preferably proceeds as normal. The more buffer is added, the longer the second reaction is delayed. If too much buffer is added, the second crosslinking reaction step may proceed too slow, and the product is not hardened enough yielding it for example unsuitable for further applications or selling of the product.

Thus the amount of buffer added depends on the desired process time and the allowable time required for hardening after production of the material.

In one embodiment at least one additional solution is applied onto the substrate, e.g. the substrate is coated together with the gelatin solution to which crosslinking compound is added. The additional solution has a pH that is lower than the pH of the gelatin solution(s) in which crosslinking compound was added. During and after coating, the layers are in contact and the remaining buffer capacity in the layer in which crosslinking compound is added will be reduced by migration of protons trough the coated layers as a result of diffusion.

Polymers that can be crosslinked with pH dependent crosslinking compounds such as for example triazines are those polymers that have hydroxyl-, amine- or thiol groups. Such polymers can be selected from the group comprising polyvinylalcohol homo-polymers or co-polymers with for examples polyvinyl acetate or polyvinyl chloride, cellulose, polyvinyl amine, polypropylene amine, gelatin or collagen. In a preferred embodiment the polymer that is crosslinked is gelatin or collagen.

As gelatin, any type of gelatin as is known in the art can be used. This comprises lime processed (bone) gelatin, acid processed (skin) gelatin, gelatin prepared from cold-blooded animals such as fish but also may be prepared by recombinant methods as described for example in EP 0926543.

The gelatin also comprises modified gelatins such as oxidized gelatin (in which a methionine group in the gelatin molecule is oxidized with e.g. hydrogen peroxide), amino group-modified gelatin (e.g., phthalated gelatin, trimellitated gelatin, succinated gelatin, maleated gelatin, and esterified gelatin).

Gelatin also comprises hydrolyzed gelatins having an average molecular weight of less than about 60 kilodaltons. Gelatin and collagen are both manifestations of the same original protein. The terms are sometimes used to indicate distinct species. With respect to hardening reaction described here such a distinction is not made. The invention can be applied for gelatin as well as collagen. It will be clear to a person skilled in the art that buffering a triazine crosslinking reaction can also be applied to other polymers than collagen or gelatin, the only requirement being the presence of free amine, thiol or hydroxy groups that can react with a crosslinking compound.

Preferably, the gelatin concentration in the coating solution to which crosslinking compound is added is between 3 and 30 weight percent, more preferably between 5 and 20 weight percent and most preferably from about 7 to 12 weight percent. Crosslinking compound can be added into one coating solution or can be divided over more coating solutions. Hence a substrate can be coated with one layer of crosslinked gelatin (or polymer to be crosslinked) or with multiple layers of crosslinked gelatin (or polymer to be crosslinked), which multiple layers may be identical, similar or different in composition, in particular with respect to polymer to be crosslinked and crosslinking compound. Preferably the crosslinking compound is added in one layer. The coating solution in which the crosslinking compound is added preferably contains the total amount of crosslinking compound necessary to harden all polymer (gelatin or collagen) containing layers after coating. Preferably the ratio of crosslinking compound to polymer, preferably gelatin or collagen, in the coating solution is at least 200 micromol crosslinking compound per gram polymer, preferably gelatin or collagen. More preferably the ratio is at least 500 micromol. Preferably the ration of crosslinking compound to polymer, preferably gelatin or collagen is at least 1 millimol crosslinking compound per gram polymer. The ratio of crosslinking compound to polymer, preferably gelatin or collagen, after coating is preferably between 5 and 200 micromol crosslinking compound per gram polymer, preferably gelatin or collagen, preferably between 25 and 150 micromol crosslinking compound per gram polymer, preferably gelatin or collagen and even more preferably from about 50 to 100 micromol crosslinking compound per gram polymer, preferably gelatin or collagen. The ratio of crosslinking compound to gelatin ratio is calculated from the total amount of crosslinking compound and the total amount of gelatin, which total amounts are found by adding up the coated amounts in each layer of the same area. The ratios of crosslinking compound to polymer, preferably gelatin or collagen, are estimated from hardening with 2-hydroxy-4,6-dichloro-s-triazine. When less reactive crosslinking compounds are used, the amounts of crosslinking compound needed to have a comparable degree of crosslinking within the same time will increase, which is a matter of routine experimentation for the skilled person.

The amount of crosslinking compound that is used further depends on the use or application of the hardened layer. Gelatin layers or matrixes are used in many image recording materials such as photographic image recording materials, inkjet recording materials, planographic recording materials and the like. Other applications using gelatin matrices are medical or pharmaceutical formulations in which gelatin sheets, sponges or particles are formed by crosslinking a gelatin matrix with a triazine crosslinking compound.

Gelatin matrices can be formed by known coating methods, such as for example slide bead coating, curtain coating, bar coating, extrusion coating or cast coating.

In one aspect the invention also concerns a sheet material, optionally on a substrate, that is obtained by the method as described above. In yet a further aspect the invention also concerns a sheet material, optionally on a substrate, that is obtainable by the method as described above. In a particular aspect the invention concerns A sheet material, optionally on a substrate, comprising

(i) a polymer that is crosslinked by a crosslinking compound, said polymer comprising prior to being crosslinked at least one group selected from a hydroxy-group a thiol-group and an amine-group and wherein said crosslinking compound crosslinks said polymer via at least one group selected from said hydroxy-group, thiol-group and amine-group of said polymer, and
(ii) a buffering compound that does not react with the crosslinking compound and has a pKa value at 25° C. of between at least 6.5 and at most 8.5 wherein said buffering compound is selected from the group consisting of structure I

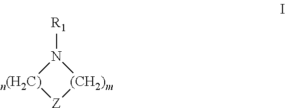

wherein
Z=carbon, oxygen, sulphur or N—R₂
2≦(n+m)≦6
R₁, R₂=—(—CH₂—)_{q}-A
A=hydrogen, —SO₃H, —COOH, —PO₃H₂ or a salt thereof
A is other than hydrogen for at least one of R1 and R2 in structure I, and
q=1-4 and if A=—COOH then q=0-4.
and structure II

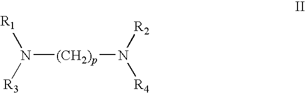

wherein
R₁, R₂, R₃ and R₄=—(—CH₂—)_{q}-A
A=hydrogen, —SO₃H, —COOH, —PO₃H₂ or a salt thereof
A is other than hydrogen for at least one of R₁, R₂, R₃ and R₄
for at least two of R₁, R₂, R₃ and R₄ A is other than hydrogen
q=1-4 and in case A=—COOH then q=0-4
1≦p≦3.

In one embodiment the sheet material comprises a buffering compound that has structure I as described above. In a preferred embodiment the sheet material comprises a buffering compound selected from 3-(N-morpholino)propanesulfonic acid and piperazine-N,N′-bis(2-ethanesulfonic acid) or a combination thereof. In another embodiment the buffering compound comprised in the sheet material is a phosphate. In yet a further embodiment the buffering compound is present in the sheet material in an amount of at least 0.01 mol per mol crosslinking compound. In yet a further embodiment the amount of buffering compound in the sheet material is between 0.1 and 0.4 mol per mol crosslinking compound. In one embodiment the polymer that is crosslinked in the present material is gelatin or collagen.

For photographic applications the ratio of crosslinking compound to gelatin is preferably between 25 and 150 micromoles crosslinking compound per gram gelatin. The final degree of hardening is tuned to the development process, which occurs at temperatures of 35° C. and higher. The gelatin layers should be hardened enough so that the gelatin does not dissolve in the development process solutions. The degree of hardening should also be high enough to resist physical damage due to for example scratching. The amount of crosslinking compound added should also be controlled within narrow specifications because the swelling rate of a hardened gelatin layer in the process solutions is a crucial parameter in the development process.

For inkjet applications the preferred crosslinking compound/gelatin ratio is between 5 and 150 micromoles crosslinking compound per gram gelatin. The final product can be subjected to higher temperatures due to environmental conditions. On a hot day the gelatin layer on an inkjet recording material should not melt. In case of inkjet the gelatin layer can serve to accommodate the solvent form the ink applied on the medium. To do that, the gelatin layer must be able to swell. Thus an image recording material comprising a sheet material according to the invention is another aspect of the present invention.

In case of artificial skin the preferred crosslinking compound/gelatin ratio is between 20 and 200 micromoles crosslinking compound per gram gelatin. The structural integrity of the gelatin sheet is also an issue, since it is applied at a temperature of more than 30° C. Artificial skins are resorbed by the (human) body by proteolysis of the gelatin. The speed with which enzymatic breakdown of the artificial gelatin occurs can be regulated by controlling the amount of crosslinking compound. Thus a tissue engineering material comprising a sheet material according to the invention is another aspect of the present invention.

The pH of the coating solution to which crosslinking compound is added is preferably between about 6.5 and 8.5, more preferably between about 7 and 8.5 or between about 7 and about 8. Higher pH values are preferred, since this slows down the second reaction step. Too high a pH of higher than 9.0 is not preferred. At high pH, the first reaction step proceeds fast, resulting in quick monolinking of the crosslinking compound molecules; at too high pH all available lysines are monolinked to crosslinking compound, so that no crosslinks can be formed.

At higher pH, such as higher than 9, also other effects start to play a role. One effect is that gelatin hydrolysis can occur in the liquid phase, reducing the time span in which the coating solution can be used. For example, especially in photographic applications in which many layers are coated in one coating run, the viscosity balance between the layers is important. Also, the gelatin solutions generally contain additives which can react or decompose at higher pH values.

Too low pH during the liquid phase will result in too high or too low reaction rate, depending on the type of crosslinking compound added. Too high reaction rate results in formation of microgels in dead spaces of the process equipment. Too low reaction rate results in longer hardening time of the product. Too low reaction rate after coating can result in sticky surfaces so that it takes longer before the product can be processed further, or can result in material that is unstable because the hardening reaction is still proceeding. In case of photographic materials in which s-triazine derivatives are used as crosslinking compound a finished product is kept under controlled temperature/humidity conditions for at least a week, waiting for the crosslinking compound reaction to approach equilibrium. Longer storage times result in a less flexible delivery schedule.

In one embodiment gelatin containing layers are coated in photographic image recording materials. Generally, between about 6 and 21 layers are simultaneously coated on a substrate using for example a slide bead coater. The basic technologies for producing photographic recording materials are described in Research Disclosure RD365044, and in for example WO-A1-2004/081661.

Crosslinking compound, preferably 2-hydroxy-4,6-dichloro-s-triazine can be added in one or more layers. Preferably crosslinking compound is not added in layers containing silverhalide crystals. Also crosslinking compound is preferably not added in layers containing an acid processed gelatin.

Preferably the buffer that is added in the layer(s) in which crosslinking compound is also added is not a phosphate buffer. Phosphate buffers can form precipitates with for example calcium ions.

Buffers for use with s-triazine crosslinking compounds preferably do not contain hydroxyl groups or amine groups or thiol groups. Preferred buffers are buffers of structure I described above. Preferably Z is sulfur or oxygen or nitrogen or N—R₂. For each nitrogen one of the attached rest groups is other than hydrogen. Preferably for the rest groups —(—CH₂—)_{q}-A, the variable ‘A’ is —SO₃H or a salt thereof.

Addition of a buffer in an embodiment according to the invention means that the pKa of the buffer is equal to or lower than the pH of the layer in which the buffer is added. Preferably the pH of the layer is 0.5-1.0 pH units higher than the pKa, so that the buffer capacity is optimally used. The buffering capacity of the added inventive buffer should not be too high. The crosslinking compound reaction is delayed in the liquid phase, before coating the crosslinking compound- and gelatin containing layer. As soon as the layer (or layers) is (are) coated onto a substrate the buffer capacity is preferably used up completely and the hardening reaction proceeds at the original higher speed. It is impossible to give the exact amount of buffer that should be added for each possible coating solution recipe. In practice the buffering capacity may be used up just before or just after coating the layer(s). It can be determined easily if the buffer capacity was not too high, by measuring the degree of crosslinking in time. The degree of hardening can be determined by measuring how much a coated layer, or coated layers, swells in an aqueous solution or in water. Such a method is described for example by Flynn and Levine (Photogr. Sci. Eng., 8, 275 (1964). Swelling is a very relevant parameter for photographic recording media, since the degree of swelling determines the rate of development of the photographic image.

To avoid too slow crosslinking speed after coating the layers, the pH of the layer(s) in which no crosslinking compound is added can optionally be decreased. After coating small components in all layers, like crosslinking compound molecules, buffer and protons will diffuse, and any excess buffer capacity will be used up immediately.

The amount of buffer needed to avoid formation of microgels in the coating equipment, and the optionally necessary pH reduction of preferably adjacent layers can easily be determined by the skilled person by a simple series of experiments.

In another embodiment the buffer is added to a gelatin solution suitable for coating a substrate for inkjet applications. The production method can be compared to that of photographic recording materials. Also for inkjet applications a too high excess of buffer capacity may not be desirable, although the demands for reproducible swelling may not be as strict as for photographic applications. Swelling in inkjet recording materials serves to accommodate high solvent loads from densely printed areas. Swelling facilitates the uptake of solvent. Also for inkjet applications the pH of other layers, preferably adjacent layers, is optionally decreased to counter the remaining buffer capacity after coating.

In yet another embodiment crosslinked gelatin containing layers are prepared for use in tissue engineering or for use as test substrate for pharmaceutical or cosmetic tests in which such a gelatin layer or membrane mimics skin. Such materials are described in co-pending PCT/NL2005/000261.

Suitable substrates are substrates having a resin surface such as a polyolefin layer. Preferably the resin layer comprises a polyethylene (PE) or polypropylene (PP), which can be a high density, a low density, a linear low density, a metallocene PE or PP or a mixture thereof. The substrate can also be a paper base coated with a resin layer.

In case the coated gelatin layer should be removed from the substrate prior to use, the resin surface is optionally subjected to an adhesion promoting treatment such as a flame treatment, a corona treatment or a plasma treatment of at least 1.5 watt·minute per square meter, preferably at least 2.5 watt·minute per square meter, and at most 30 watt·minute per square meter, preferably at most 25, 20, 15, 10 or 5 watt·minute per square meter before coating. Purpose of the adhesion promoting treatment is to provide enough adhesion so that the material can be coated, dried and subjected to processes such as rolling up or cutting without release of the non-porous film. On the other hand the adhesion should be weak enough to facilitate easy separation from the substrate prior to use.

### DESCRIPTION OF THE FIGURES

FIG. 1 shows the effect of pH and of amount of buffer added on the hardening speed of gelatin.

FIG. 2 shows the relation between hardening speed and amount of buffer.

FIG. 3 is a comparison of the effect of different buffers according the invention; at t=0, pH=7.1 and 0.268 mol buffer/mol hardener was used.

### EXAMPLES

### Example 1

### Effect of pH and Buffer Addition on Hardening Rate of a Gelatin Solution

A solution for coating a UV-light protective layer as described in WO-A1-2004081661 was prepared with the following composition:

Lime bone gelatin: 83.3 gram per 1000 ml solution
oil: 34.8 gram per 1000 ml solution
ethylacetate: 9.9 gram per 1000 ml solution
UV-absorber: 54.1 gram UV-absorbing compound per 1000 ml solution
Cyan dye: 0.59 gram per 1000 ml solution
add water to 1000 ml.

The solution was prepared by adding gelatin into a cold water solution, and allow to swell. After swelling the temperature was increased to 40° C. to dissolve the gelatin. The UV-absorbing compounds were dissolved in the oil after addition of the ethylacetate. After addition of a surfactant the oil phase was dispersed into the gelatin solution.

The cyan dye was added as a 5% aqueous solution.

Finally a buffer compound was added according table 1 and where necessary the pH was adjusted with a base or acid to the values as shown in table 1. As bases hydroxides can be used. Acids can be hydrochloric acid, citric acid, sulphuric acid. Any other base or acid can be used as long as it does not have detrimental effects in the hardening process.

To this solution an 8% crosslinking compound solution was added, stabilized with approximately 0.18 mol carbonate/bicarbonate per mol crosslinking compound. After addition, the gelatin concentration of the coating solution was about 7.5 weight percent.

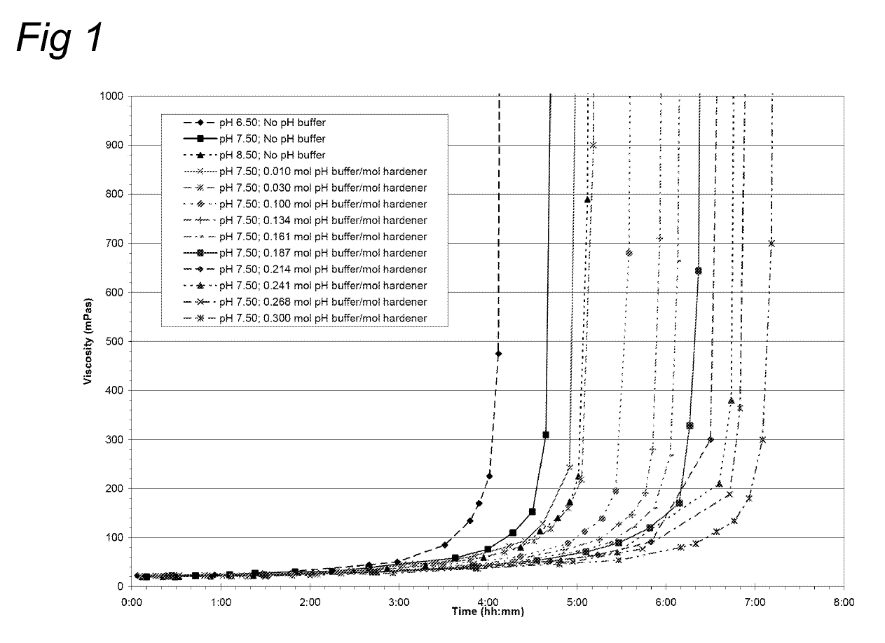

As a measure for the hardening speed, the hardening time to reach a viscosity of 1000 cp, as shown in FIG. 1, is taken.

TABLE 1

| | | | | hardening |
|---|---|---|---|---|
| | | additional | amount (mol/mol | time |
| | pH | buffer | crosslinking compound) | (minutes) |
| comp 1 | 6.5 | none | 0 | 250 |
| comp 2 | 7.5 | none | 0 | 280 |
| inv 1 | 7.5 | MOPS/pKa 7.3 | 0.01 | 299 |
| inv 2 | 7.5 | MOPS/pKa 7.3 | 0.03 | 311 |
| inv 3 | 7.5 | MOPS/pKa 7.3 | 0.10 | 336 |
| inv 4 | 7.5 | MOPS/pKa 7.3 | 0.13 | 356 |
| inv 5 | 7.5 | MOPS/pKa 7.3 | 0.16 | 369 |
| inv 6 | 7.5 | MOPS/pKa 7.3 | 0.19 | 383 |
| inv 7 | 7.5 | MOPS/pKa 7.3 | 0.21 | 394 |
| inv 8 | 7.5 | MOPS/pKa 7.3 | 0.24 | 406 |
| inv 9 | 7.5 | MOPS/pKa 7.3 | 0.27 | 413 |
| inv 10 | 7.5 | MOPS/pKa 7.3 | 0.30 | 432 |
| comp 3 | 8.5 | none | 0 | 310 |

The comparative examples show that the traditional measure, that is increase of starting pH, delays the hardening reaction to some extent. However even addition of a small amount of buffer according to the invention (inv 1, 2) has an effect similar to increasing the pH by a full unit.

Emulsions in which crosslinking compound is added can contain further additives that may be pH-sensitive. Increasing pH is therefore not always an option. In such cases adding even a small amount of buffer may sufficiently delay the hardening reaction.

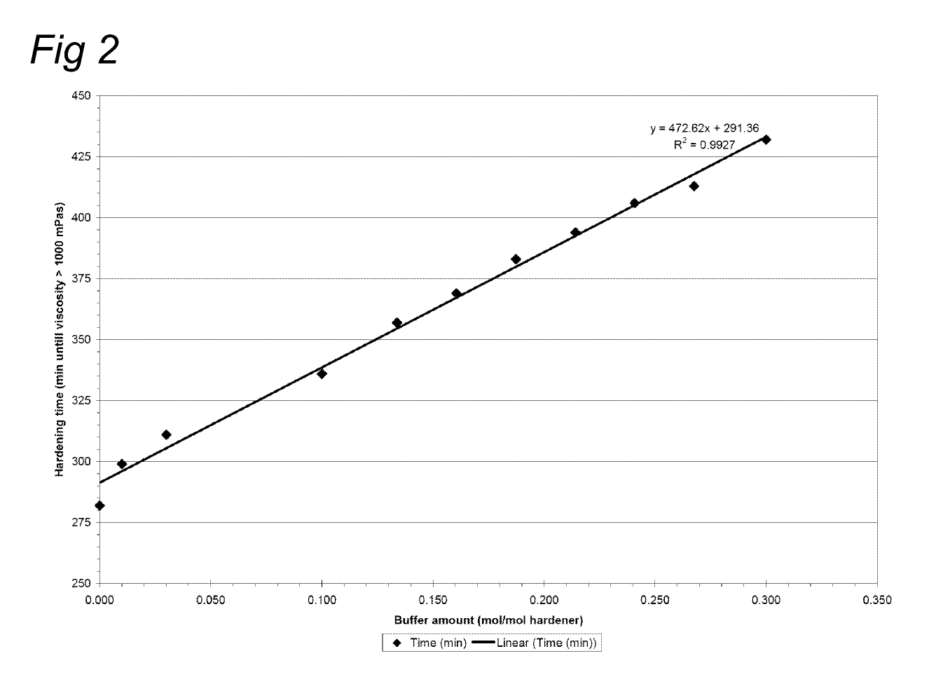

In FIG. 2 the hardening speed, expressed as the time necessary to reach viscosity of 1000 cp, is plotted as a function of the amount of buffer added. This results in a linear plot. In practice the balance between delaying the crosslinking compound speed (prevent forming microgels in coating equipment) and limiting the amount of buffer added (prevent too slow hardening after coating the layer(s)) is determined experimentally. We found that thanks to the linearity of the relation between hardening speed and amount of buffer added, the optimal amount of buffer that can be added can be easily predicted by a person skilled in the art.

### Example 2

### Comparison of Different Buffers with Comparable pKa Values

A series experiments was carried out in the same way as in example 1. Three different buffers were tested with comparable pKa's as shown in table 2:

TABLE 2

| | | | | hardening |
|---|---|---|---|---|
| | | additional | amount (mol buffer/mol | time |
| | pH | buffer | crosslinking compound) | (minutes) |
| comp | 7.1 | none | not applicable | 240 |
| inv | 7.1 | Na₂HPO₄/pKa 7.2 | 0.268 | 475 |
| inv | 7.1 | MOPS/pKa 7.3 | 0.268 | 450 |
| inv | 7.1 | PIPES/pKa 7.1 | 0.268 | 440 |

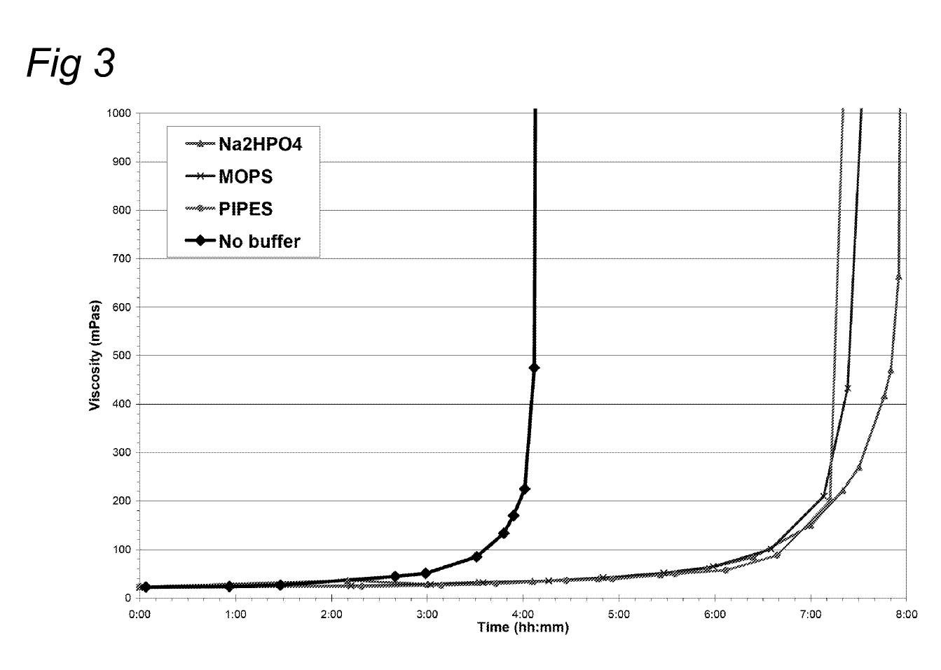

All three buffers delay the hardening reaction in a similar way as can be seen in FIG. 3. From comparing the phosphate buffer to the organic MOPS and PIPES buffers it can be concluded that the choice of pKa is more important than the nature of the buffer.

In practice inorganic buffers may be less preferred since these more easily can form precipitates such as for example calciumphosphates.

1. A method for producing a sheet material comprising at least one crosslinked polymer layer on a substrate, said method comprising the steps of:
(a) providing a coating solution comprising between 3 and 30 weight percent of a dissolved polymer that comprises at least a hydroxy group, a thiol group, or an amine group, and a buffering compound that does not react with the crosslinking compound of (c), and has a pKa value of between 6.5 and 8.5 at 25° C.;
(b) adjusting the pH of said coating solution to a pH, at 40° C., of between 6.0 and 9.0;
(c) adding a crosslinking compound to said coating solution; and
(d) applying said coating solution onto said substrate within 30 minutes of adding said crosslinking compound, to obtain a coated layer; and

2. The method according to claim 1 wherein the coating solution obtained in step (c) has the following property: |(pH_{40° C.}−pKa_{25° C.})|≦1.0.

3. The method according to claim 1 wherein the crosslinking compound is a triazine type crosslinking compound.

4. The method according to claim 3 wherein the crosslinking compound is 2-hydroxy-4,6 dichlorotriazine.

5. The method according to claim 1, wherein the buffering compound is
(a) a compound of Formula I: 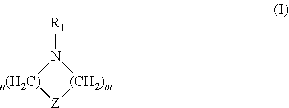
wherein:
Z is carbon, oxygen, sulfur or N—R₂.
2≦(n+m)≦6,
R₁ and R₂ are (CH₂)_{q}A, wherein
A is hydrogen, —SO₃H, —COOH, —PO₃H₂ or a salt thereof, and
A is other than hydrogen for at least one of R₁ and R₂, and
q is 1-4. except,
when A is —COOH, q is 0-4; or
(b) a compound of Formula II: 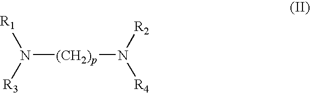
wherein
R₁, R₂, R₃ and R₄ are (CH₂)_{q}A, wherein
A is hydrogen, —SO₃H, —COOH, —PO₃H₂ or a salt thereof,
A is other than hydrogen for at least one of R₁, R₂, R₃ and R₄
q is 1-4, except when A is —COOH,
q is 0-4, and
1≦p≦3.

6. The method according to claim 1 wherein said buffering compound is a phosphate.

7. The method according to claim 1, wherein the amount of said buffering compound is at least 0.01 mole per mole of said crosslinking compound.

8. The method according to claim 1, wherein said dissolved polymer being crosslinked is gelatin or collagen.

9. A sheet material, comprising
(a) a polymer that is crosslinked by a crosslinking compound, which polymer, prior to being crosslinked, comprises at least a hydroxy group a thio group or an amine group, and wherein said crosslinking compound crosslinks said polymer via said hydroxy-group, said thio group or said amine group,
(b) a buffering compound that does not react with the crosslinking compound and has a pKa value at 25° C. of between 6.5 and 8.5 wherein said buffering compound is
(i) a compound of Formula I 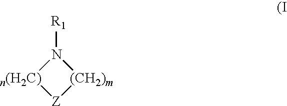
wherein:
Z is carbon, oxygen, sulfur or N—R₂.
2≦(n+m)≦6,
R₁ and R₂ are (CH₂)_{q}A, wherein
A is hydrogen, —SO₃H, —COOH, —PO₃H₂ or a salt thereof, and
A is other than hydrogen for at least one of R₁ and R₂, and
q=1-4. except,
when A is —COOH, q is 0-4; or
(ii) a compound of Formula II: 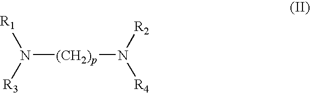
wherein
R₁, R₂, R₃ and R₄ are (CH₂)_{q}A, wherein
A is hydrogen, —SO₃H, —COOH, —PO₃H₂ or a salt thereof,
A is other than hydrogen for at least one of R₁, R₂, R₃ and R₄
q is 1-4, except when A is —COOH,
q is 0-4, and
1≦p≦3.

10. The sheet material according to claim 9, wherein said buffering compound is 3-(N-morpholino)propanesulfonic acid, piperazine-N,N′-bis(2-ethanesulfonic acid) or a combination thereof.

11. The sheet material according to claim 9, wherein the buffering compound is a phosphate.

12. The sheet material according to claim 9, wherein the amount of said buffering compound is at least 0.01 mole per mole crosslinking compound.

13. The sheet material according to claim 9, wherein the amount of said buffering compound is between 0.1 and 0.4 mole per mole crosslinking compound.

14. The sheet material according to claim 9, wherein said polymer being crosslinked is gelatin or collagen.

15. An image recording material comprising a sheet material according to claim 9.

16. A tissue engineering material comprising a sheet material according to claim 9.

17. A method according to claim 1 further comprising the step of:
(e) separating said substrate from said coated layer.

18. The method according to claim 5 wherein, when R₁, R₂, R₃ and R₄ are (CH₂)_{q}A,
is hydrogen, —SO₃H, —COOH, —PO₃H₂ or a salt thereof,
wherein, for at least two of R₁, R₂, R₃ and R₄, A is other than hydrogen.

19. The sheet material according to claim 9, wherein, when R₁, R₂, R₃ and R₄ are (CH₂)_{q}A,
A is hydrogen, —SO₃H, —COOH, —PO₃H₂ or a salt thereof,
wherein, for at least two of R₁, R₂, R₃ and R₄, A is other than hydrogen.

20. The sheet material according to claim 9 which is on a substrate.
